(19) United States
(12) Patent Application Publication
(10) Pub. No.: US 2021/0137898 A1
(43) Pub. Date: May 13, 2021

(54) METHODS TO TREAT GLIOMAS USING A STAT3 INHIBITOR

(71) Applicant: Mayo Foundation for Medical Education and Research, Rochester, MN (US)

(72) Inventor: David J. Daniels, Rochester, MN (US)

(73) Assignee: Mayo Foundation for Medical Education and Research, Rochester, MN (US)

(21) Appl. No.: 16/621,804

(22) PCT Filed: Jun. 15, 2018
(86) PCT No.: PCT/US2018/037772

§ 371 Date: Dec. 12, 2019

Related U.S. Application Data
(60) Provisional application No. 62/520,463, filed on Jun. 15, 2017.

(51) Int. Cl. A61K 31/44; A61K 9/00; A61K 31/196; A61K 31/4155; A61K 31/415; A61P 35/00

(52) U.S. Cl. CPC A61K 31/44; A61K 9/0053; A61P 35/00; A61K 31/4155; A61K 31/415; A61K 31/196

(57) ABSTRACT

The present application provides a method to treat a glioma in a subject, such as Diffuse Intrinsic Pontine Glioma (DIPG), using at least one STAT3 inhibitor. The glioma treatable with a STAT3 inhibitor may have a mutation in a histone H3 gene, including H3F3A. For example, the glioma may have a H3K27M mutation. Suitable examples of STAT3 inhibitors include WP1066, S3I-201 and C1-C10, or a pharmaceutically acceptable salt thereof.

### CLAIM OF PRIORITY

This application claims the benefit of U.S. Provisional Application No. 62/520,463, filed Jun. 15, 2017. The entire contents of the foregoing are hereby incorporated by reference.

### TECHNICAL FIELD

This invention relates to compositions and methods to treat gliomas, such as diffuse midline gliomas, using at least one inhibitor of STAT3 pathway. In particular, the invention relates to treating gliomas having mutations in histone H3 genes, including H3F3A. Examples of the histone mutations include H3K27M and H3K27I. The invention also relates to treating gliomas (e.g., midline gliomas) having hypomethylation of H3K27me3.

### BACKGROUND

The signal transducer and activator of transcription (STAT) proteins are considered a family of transcriptional factors that are activated in response to growth factors and cytokines and promote cell proliferation and survival (Yu, et al. Crosstalk between cancer and immune cells: role of STAT3 in the tumour microenvironment. Nat Rev Immunol. 2007; 7, 1:41-51). Extracellular signals can activate Janus kinases (JAKs) and receptor tyrosine kinases that in turn activate STATs by phosphorylating a critical tyrosine residue in the active site. A promising location for STAT3 inhibition could be the Src Homology 2 (SH2) domain of STAT3, inhibiting the STAT3 molecule by directly preventing phosphorylation of STAT3, or preventing active phospho-STAT3 homodimer formation. Two phosphorylated STAT monomers are believed to form a homodimer that translocates to the nucleus to bind specific DNA-response elements in the promoters of target genes and induce gene expression (Yu, et al. Enhanced DNA-binding activity of a Stat3-related protein in cells transformed by the Src oncoprotein. Science. 1995; 269, 5220:81-83).

### SUMMARY

In some embodiments, the present application provides a method of treating a malignant glioma in a subject, the method comprising: a) identifying a mutation in a histone H3 gene in a glioma cell obtained from the subject; and b) after a), administering to the subject a therapeutically effective amount of a STAT3 inhibitor, or a pharmaceutically acceptable salt thereof.

In some embodiments, the glioma is pediatric.

In some embodiments, the glioma is a high-grade glioma (HGG).

In some embodiments, the glioma is a midline glioma.

In some embodiments, the glioma is a diffuse midline glioma.

In some embodiments, the glioma is a thalamic, brainstem, or upper spine glioma.

In some embodiments, the glioma is Diffuse Intrinsic Pontine Glioma (DIPG).

In some embodiments, the histone H3 gene is H3F3A.

In some embodiments, the mutation leads to an amino acid substitution in the histone tail.

In some embodiments, the amino acid is lysine (K).

In some embodiments, the lysine is substituted with a methionine (M).

In some embodiments, the mutation in the histone H3 gene results in a translation of a H3 histone having a K27M amino acid substitution (H3K27M mutation).

In some embodiments, the lysine is substituted with an isoleucine (I).

In some embodiments, the mutation in the histone H3 gene results in a translation of a H3 histone having a K27I amino acid substitution (H3K27I mutation).

In some embodiments, the mutation leads to global hypomethylation of H3 histones in the glioma.

In some embodiments, the mutation leads to decreased levels or global loss of H3K27me3 and/or H3K27me2 in the glioma.

In some embodiments, the present application provides a method of treating midline gliomas with the H3K27M mutation in a subject, the method comprising administering to the subject a therapeutically effective amount of a STAT3 inhibitor, or a pharmaceutically acceptable salt thereof.

In some embodiments, the present application also provides a method of treating Diffuse Intrinsic Pontine Glioma (DIPG) in a subject, the method comprising administering to the subject a therapeutically effective amount of a STAT3 inhibitor, or a pharmaceutically acceptable salt thereof.

In some embodiments, the Diffuse Intrinsic Pontine Glioma (DIPG) is pediatric.

In some embodiments, the STAT3 inhibitor, or a pharmaceutically acceptable salt thereof, is administered to the subject orally.

In some embodiments, the STAT3 inhibitor, or a pharmaceutically acceptable salt thereof, is a blood brain barrier penetrant.

In some embodiments, the STAT3 inhibitor is WP1066 having the following structure:

[chemical structure]

or a pharmaceutically acceptable salt thereof.

In some embodiments, the STAT3 inhibitor is S3I-201 having the following structure:

[chemical structure]

or a pharmaceutically acceptable salt thereof.

In some embodiments, the STAT3 inhibitor is C10 having the following structure:

[chemical structure]

or a pharmaceutically acceptable salt thereof.

In some embodiments, the STAT3 inhibitor is selected from any one of the following compounds (C1-C9):

[chemical structure]

or a pharmaceutically acceptable salt thereof.

In some embodiments, the STAT3 inhibitor directs dephosphorylation and nuclear export of constitutively phosphorylated STAT3.

In some embodiments, the administration of STAT3 inhibitor to the subject leads to an increased level of a methylated H3 histone in the glioma.

In some embodiments, the methylated H3 histone is H3K27me2 and/or H3K27me2.

In some embodiments, the increased level of the methylated H3 histone results in the treatment of the glioma in the subject.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1 shows that Wnt5a is essential for survival of DIPG cells with H3K27M mutation. Depletion of Wnt5a inhibits proliferation of DIPG cells (SF7761 and SF8628, top panels), but not WT control (SF9427) or H3G34V (KNS42). Depletion of β-catenin by two different shRNAs had no apparent effect on these lines. NT is scrambled control. Western blot analysis of Wnt5a and β-catenin indicated that these proteins were effectively depleted (data not shown).

FIG. 2 shows that orthotopic xenografts with Wnt5a knockdown show reduced tumor growth compared to controls. Bioluminescence imaging (BLI) was obtained using IVIS Lumina imaging system using luciferase transfected tumor cells. In the H3K27M tumor cell line (SF7761), shRNA depletion decreased tumor growth compared to scrambled shRNA vector or non-treated tumor cells. Cohorts of mice (n=9 or 10/group), were implanted in the brainstem and imaging was performed weekly. Week 20 shown.

FIG. 3 shows identifying STAT3 as a downstream effector involved in Wnt5a signaling in DIPG cells. A. Using the QiAGEN 45 Cignal Finder reporter assay, H3K27M tumor cells (SF8628) were treated with shRNA against Wnt5a or non-target control. Results of only 21 reporters shown to save space. The only reporter to decrease after Wnt5a knockdown was STAT3. B. Depletion of Wnt5a affects Stat3 phosphorylation in H3K27M lines (SF7761, SF8628) but not controls (39RG2 and KNS42).

FIG. 4 shows that A. STAT3 is critical for cellular proliferation in H3K27M tumors vs. WT Gliomas Depletion of Stat3 using two different shRNAs (middle and right bars) normalized to scrambled shRNA vector (NT, left bar)—results in reduced cell viability of DIPG cells. Results are mean±SD of three independent experiments. B and C. H3K27M cells (Peds8 and DIPG8) are selectively sensitive to inhibition by STAT3 inhibitors. [B. WP1066: Peds8, IC50=1.9 μm; DIPG8, IC50=2.5 μm; vs. WT SF9427 IC50=62.3 μm. C. DJDLeu: Peds8, IC50=1.7 μm; DIPG8, IC50=3.3 μm; vs. WT SF9427 IC50=25.4 μm].

FIG. 5 shows that STAT3 and pSTAT3 expression is elevated in patient tumors with the H3K27M mutation. A. Surgical samples prior to treatment show significantly higher levels of pSTAT3 in DIPG tumors compared to normal brain (removed during other surgeries). When normalized for Tubulin, pSTAT3 levels are >20-fold higher in DIPG tumors. B. Published datasets show patients with DIPG tumors have greatly increased STAT3 expression by RNA-seq compared to normal brain (p<0.0001).

FIG. 6 shows that STAT3 expression is high in H3K27M cell lines and treatment with STAT3 pathway inhibitors increase global H3K27 trimethylation. A. Basal pSTAT3 levels are generally increased in H3K27M DIPG tumors vs. WT tumors including 2 Adult GBMs and 1 Pediatric GBM. B-D. Treatment of H3K27M tumors cells (Peds8) with STAT3 Inhibitor WP1066 (B) and JAK2 Inhibitor Ruxolitinib (C) increases H3K27me3 similar to the histone demethylase inhibitor GSKJ4 (D, NT is no treatment and represents basal H3K27M hypomethylation). Inhibiting STAT3 Signaling Increases H3K27me3 in DIPG Tumor Cells.

FIG. 7 shows that the STAT3 inhibitor WP1066 reduces H3K27M tumor growth in patient derived orthotopic xenografts. H3K27M tumor Peds17 treated with STAT3 inhibitor WP1066 (20 mg/kg oral gavage) vs. control (DMSO) for 8 weeks. Significant decrease in tumor growth observed with WP1066 vs. control (p=0.03). Cohorts of mice (n=10/group), were implanted in the brainstem and BLI was performed weekly using IVIS Lumina imaging system using luciferase transfected tumor cells.

FIG. 8 shows that depletion of Wnt5a in two DIPG lines results in reduced ability to form colonies.

FIG. 9 shows that depletion of Wnt5a leads to apoptosis of two DIPG lines with H3.3K27M mutation.

FIG. 10 shows Wnt5a Gene Expression—From Published Datasets.

FIG. 11 is a diagram showing STAT3 pathway.

FIG. 12 is a Western blot showing proteins in patient plasma.

FIG. 13 is a diagram showing Wnt5a pathway.

FIG. 14 shows a gene map.

FIG. 15 shows FDA drug screen, most potent class of compounds.

FIG. 16 shows STAT3 Reporter Assay with FDA TKIs.

FIG. 17 shows Western blot showing STAT3 and pSTAT3 proteins bands.

FIG. 18 is a diagram showing Wnt5a and STAT3 pathways.

### DETAILED DESCRIPTION

Midline gliomas (e.g., diffused midline gliomas) with the H3K27M mutation, including the previously named diffuse intrinsic pontine gliomas (DIPG), are the most aggressive primary malignant brain tumors in children with the median survival after diagnosis being about one year with no effective therapies available.¹⁻⁴ Groundbreaking studies have recently found a somatic mutation of the H3F3A gene that encodes the histone H3 variant, H3.3, which results in lysine 27 to methionine change (K27M) in the encoded protein H3.3 in most DIPG tumors.⁵⁻⁷ Understanding how the H3K27M mutation promotes tumorigenesis is important and can enable discovery of novel targets that are critical to survival and proliferation of these tumors. The H3K27M mutation (H3M27 proteins) drives the global loss of di- and trimethylation of histone H3K27 (H3K27me2 and H3K27me3) on wild type histone proteins.⁸⁻¹¹ This phenotypic hallmark of midline gliomas (e.g., diffuse midline gliomas) is observed in greater than 95% of these tumors.^{12, 13} In the largest cohort of classic DIPG patients that underwent a biopsy prior to treatment, 90 out of 91 patients had a H3K27M mutation or H3K27 hypomethylation driven by a similar mutation.¹⁴ The present application provides results of the experiments that shown that H3K27M mutation reprograms gene expression and histone methylation patterns, and is a key driver for these deadly tumors.^{8, 15}This mutation creates unique therapeutic vulnerabilities, which can be exploited to develop novel therapies. The present application also describes a genome wide shRNA screen and identification of two interconnected signaling pathways that are critical for survival of H3K27M tumors. The present application also shows the therapeutic efficacy of a blood brain penetrant STAT3 inhibitor, WP1066, in both xenograft and genetic engineered mouse models (GEMM) of H3K27M tumors. The results described in the present application provide insight into the molecular mechanisms that drive tumorigenesis of H3K27M tumors.

The results described herein show that two signaling pathways that are connected, Wnt5a (a protein involved in non-canonical Wnt signaling pathway) and a downstream regulator, STAT3 (an oncogenic transcription factor), and are essential for proliferation and survival of tumors with the H3K27M mutation. STAT3 inhibition was shown to be more efficacious in malignant gliomas with the H3K27M mutation than in WT gliomas.

In one general aspect, the present application provides a method of treating a glioma in a subject, the method comprising: a) identifying a mutation in a histone H3 gene of the subject (e.g., a mutation in a histone H3 gene in a glioma cell of the subject); and b) administering to the subject a therapeutically effective amount of a STAT3 inhibitor, or a pharmaceutically acceptable salt thereof. In some embodiments, the administering of step b) occurs after the identifying of step a). In other embodiments, the administering of step b) occurs prior to the identifying of step a).

In another general aspect, the present application provides a method of treating Diffuse Intrinsic Pontine Glioma (DIPG) in a subject, the method comprising administering to the subject a therapeutically effective amount of a STAT3 inhibitor, or a pharmaceutically acceptable salt thereof. In some embodiments, the subjects is in need of DIPG treatment (e.g., the subject is diagnosed with DIPG).

In some embodiments, the glioma is malignant (e.g., cancerous tumor of the brain or the spine). In some embodiments, the glioma is pediatric (e.g., the subject having the glioma is a child 0-18 years old). In some embodiments, the glioma is selected from ependymoma (e.g., intracranial, myxopapillary ependymoma, extraspinal ependymoma, or extradural ependymoma), astrocytoma (e.g., oligoastrocytoma, anaplastic astrocytoma, glioblastoma multiforme, subependymoma, subependymal giant cell astrocytoma, pleomorphic xanthoastrocytoma, or pilocytic astrocytoma), oligodendroglioma, brainstem glioma, optic nerve glioma, and oligoastrocytoma. In some embodiments, the glioma is a glioblastoma. In some embodiments, the glioma is medulloblastoma. In some embodiments, the glioma is non-brainstem glioblastoma. In some embodiments, the glioma is low-grade (LGG) or high-grade (HGG). In some embodiments, the glioma is supratentorial (e.g., above the tentorium, in the cerebrum), infratentorial (e.g., below the tentorium, in the cerebellum), or pontine (e.g., in the pons of the brainstem). In some embodiments, the glioma is thalamic. In some embodiments, the glioma is brainstem glioma. In some embodiments, the glioma is an upper spine glioma. In some embodiments, the glioma is located in the pons, midbrain or medulla. In some embodiments, the glioma is located in the hemisphere. In some embodiments, the glioma is a midline glioma. In some embodiments, the glioma is a diffuse midline glioma. In some embodiments, the glioma is Diffuse Intrinsic Pontine Glioma (DIPG). In some embodiments, the subject is in need of glioma treatment (e.g., the subject is diagnosed with a glioma).

In some embodiments, a mutation in a histone H3 gene of a subject (e.g., a mutation in a histone H3 gene in a glioma cell of the subject) may be identified without obtaining a glioma cell from the subject. For example, the mutation may be identified by analyzing a blood sample of the subject, or a sample of hair, urine, saliva, or feces of the subject. In other embodiments, a mutation in a histone H3 gene may be identified by obtaining a glioma cell from the subject (e.g., via biopsy). For example, the glioma cell for analysis of a mutation may be obtained from the patient by surgical means (e.g., laparoscopically). In these embodiments, a mutation of the H3 gene is being identified in the glioma cell of the subject.

Any of the methods, reagents, protocols and devices generally known in the art may be used to identify the mutation. In some embodiments, an assay may be used to determine whether the patient has a mutation in a histone H3 gene, using a sample from a patient. For example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR) techniques may be used to identify the mutation. As is well-known in the art, the assays are typically performed, e.g., with at least one labelled nucleic acid probe or at least one labelled antibody or antigen-binding fragment thereof. Assays can utilize other detection methods known in the art for detecting a mutation in a histone H3 gene. The sample may be a biological sample or a biopsy sample (e.g., a paraffin-embedded biopsy sample) from the patient. In some embodiments, the patient is a patient suspected of having a glioma having a mutation in a histone H3 gene (e.g., H3K27M mutation).

In some embodiments, the histone H3 gene is H3F3A or HIST1H3B. These genes are encoding histones H3, including the H 3.1 and H3.3 variants. In some embodiments, a mutation in the histone H3 gene results in at least one amino acid change in the encoded histone protein. In some embodiments, the mutation occurs in an exon encoding a lysine amino acid in a gene that encodes a histone protein. In such embodiments, the mutation in the histone H3 gene includes a recurrent somatic adenine-to-thymine transversion resulting in a substitution to methionine at lysine in the encoded histone protein. In some embodiments, the lysine is Lys27.

In some embodiments, the mutation leads to the substitution of an amino acid in the tail of the histone protein. In some embodiments, the amino acid is lysine (K), which is substituted with a methionine (M). In some embodiments, the amino acid is lysine (K), which is substituted with a isoleucine (I). In other embodiments, the amino acid is glycine (G), which is substituted with an arginine (R) or valine (V). In some embodiments, the mutation in the histone H3 gene results in a translation of a H3 histone protein having a K27M, G34R, and/or G34V amino acid substitution (e.g., the mutation is H3K27M, H3K27I, H3G34R, and/or H3G34V mutation). In some embodiments, the histone is a H3.3 isoform, and the mutation is H3.3K27M.

Histone proteins are modified post-translationally, and these post-translational modifications include acetylation, methylation, and phosphorylation. The modified (e.g., methylated) histone proteins play in important role in gene expression. A single amino acid mutation in a histone protein may alter the levels of post-translationally modified histones in a cell and reprogram the epigenetic landscape and gene expression in the cell.

In some embodiments, a mutation in a histone H3 gene leads to global hypomethylation of the wild-type histone proteins in a cell (e.g., H3 histones in a glioma cell). In some embodiments, the mutation leads to global loss of post-translational methylation products of histone H3K27. In some aspects of these embodiments, the mutation leads to decreased levels or global loss of H3K27me1, H3K27me2 and/or H3K27me3 histone proteins in a cell (e.g., glioma cell). In some embodiments, the mutated H3K27M histones affect the endogenous H3K27 methylation and the subsequent gene expression profile in a cell. In some aspects of these embodiments, the gene expression may be altered through epigenetic mechanisms including inhibiting the methylating activity of the PRC2 complex.

STAT3 Inhibitors

Signal transducer and activator of transcription 3 (STAT3) is a transcription factor playing a pivotal role in a cell signally pathway (the STAT3 pathway). The STAT3 signaling leading to expression of cellular proteins is schematically shown in FIGS. 11 and 18. In these processes, STAT3 is phosphorylated by a kinase enzyme, followed by translocation of the phosphorylated protein to the nucleus. In some embodiments, a STAT3 inhibitor directs dephosphorylation and nuclear export of constitutively phosphorylated STAT3. In other embodiments, a STAT3 inhibitor inhibits phosphorylation of STAT3. In yet other embodiments, a STAT3 inhibitor inhibits an active phospho-STAT3 homodimer formation. These embodiments are not exclusive and other mechanisms of STAT3 signaling inhibition generally known in the art may be employed.

In some embodiments, the STAT3 inhibitor is WP1066 (CAS Registry No. 857064-38-1) having the following structure:

[chemical structure]

or a pharmaceutically acceptable salt thereof.

In some embodiments, the STAT3 inhibitor is S3I-201 having the following structure:

[chemical structure]

or a pharmaceutically acceptable salt thereof.

In some embodiments, the STAT3 inhibitor is any one of pyrazole derivatives described, for example, in US application publication No. 2015/0166484. In some embodiments, the STAT3 inhibitor is a C10 compound having the following structure:

[chemical structure]

or a pharmaceutically acceptable salt thereof.

In some embodiments, the STAT3 inhibitor is selected from any one of the following compounds (C1-C9):

[chemical structure]

or a pharmaceutically acceptable salt thereof.

In some embodiments, the STAT3 inhibitor is selected from STA-21 (CAS 28882-53-3), galiellalactone (CAS 133613-71-5), auranofin (CAS 34031-32-8), 6-nitrobenzo[b]thiophene-1,1-dioxide (CAS 19983-44-9), cucurbitacin I (CAS 2222-07-3), kahweol (CAS 6894-43-5), nifuroxazide (CAS 965-52-6), S3I-201 (CAS 501919-59-1), 15-DPP (CAS 22112-89-6), niclosamide (CAS 50-65-7), cryptotanshinone (CAS 35825-57-1), cryptotanshinone (CAS 35825-57-1), STAT3 Inhibitor VII (CAS 1041438-68-9), SD 1008 (CAS 960201-81-4), and cepharanthine (CAS 481-49-2), or a pharmaceutically acceptable salt thereof.

In some embodiments, two or more of the STAT3 inhibitors may be administered to the subject. For example, the present method comprises administering to the subject WP1066 in combination with the C10 compound.

In some embodiments, the administration of STAT3 inhibitor to the subject leads to an increased level of methylated H3 histones a cell of the subject (e.g., in a cell of the subject's glioma). In these embodiments, the increased level of the methylated H3 histone induces apoptosis of the glioma cells of the subject (e.g., leads to glioma cell death and the treatment of the glioma in the subject).

Combinations

In some embodiments, the method of glioma in a subject further comprises administering to the subject an additional therapeutic agent, or pharmaceutically acceptable salt thereof. Suitable examples of additional therapeutic agents include a pain relief agent (e.g., a nonsteroidal anti-inflammatory drug such as celecoxib or rofecoxib), an antinausea agent, or an additional anticancer agent (e.g., paclitaxel, docetaxel, daunorubicin, epirubicin, fluorouracil, melphalan, cis-platin, carboplatin, cyclophosphamide, mitomycin, methotrexate, mitoxantrone, vinblastine, vincristine, ifosfamide, teniposide, etoposide, bleomycin, leucovorin, taxol, herceptin, avastin, cytarabine, dactinomycin, interferon alpha, streptozocin, prednisolone, irinotecan, sulindac, 5-fluorouracil, capecitabine, oxaliplatin/5 FU, abiraterone, letrozole, 5-aza/romidepsin, or procarbazine). In certain embodiments, the anticancer agent is paclitaxel or docetaxel. In other embodiments, the anticancer agent is cisplatin or irinotecan. In some embodiments, the method of treating cancer in a subject further comprises administering to the subject a cell carcinoma treatment. Examples of additional optional renal cell carcinoma treatments include, without limitation, treatment with Nexavar®, Sutent®, Torisel®, Afinitor® (everolimus), axitinib, pazopanib, levatinib, interleukin-2, and combinations thereof. In some embodiments, the method of treating glioma in a subject further comprises administering to the subject a proteasome inhibitor. Exemplary proteasome inhibitors include lactacystin, bortezomib, dislfiram, salinosporamide A, carfilzomib, ONX0912, CEP-18770, MLN9708, epoxomicin, and MG132). Non-limiting examples of proteasome inhibitors include marizomib (NPI-0052), bortezomib (Velcade®), and carfilzomib (Kyprolis®).

In some embodiments, the additional therapeutic agent is administered to the subject prior to the administration of the STAT3 inhibitor. In other embodiments, the additional therapeutic agent is administered to the subject after the administration of the STAT3 inhibitor. In yet other embodiments, the STAT3 inhibitor and the additional therapeutic agent are administered to the subject simultaneously (e.g., in the same dosage form or in separate dosage forms).

Pharmaceutical Compositions and Formulations

The present application also provides pharmaceutical compositions comprising an effective amount of a therapeutic compound (e.g., a STAT3 inhibitor and/or an additional therapeutic agent) disclosed herein, or a pharmaceutically acceptable salt thereof; and a pharmaceutically acceptable carrier. The pharmaceutical composition may also comprise any one of the additional therapeutic agents described. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in one embodiment 0.1-95%, in another embodiment 75-85%, in a further embodiment 20-80%, wherein the balance may be made up of any pharmaceutically acceptable excipient described herein, or any combination of these excipients.

Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, capsules (e.g., hard or soft gelatin capsules), sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspending agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the present application provides an implantable drug release device impregnated with or containing a compound or a therapeutic agent, or a composition comprising a compound of the present application or a therapeutic agent, such that said compound or therapeutic agent is released from said device and is therapeutically active.

Dosages and Regimens

In the pharmaceutical compositions of the present application, a therapeutic compound is present in an effective amount (e.g., a therapeutically effective amount).

Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of a therapeutic compound can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg).

In some embodiments, an effective amount of a therapeutic compound is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

Kits

The present invention also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers, etc. Instructions, either as inserts or as labels, indicating quantities of the components to be administered, guidelines for administration, and/or guidelines for mixing the components, can also be included in the kit. The kit may optionally include directions to perform a test to determine a mutation in a glioma cell, and/or any of the reagents and device(s) to perform such tests. The kit may also optionally include an additional therapeutic agent.

### Definitions

As used herein, the term “about” means “approximately” (e.g., plus or minus approximately 10% of the indicated value).

As used herein, the term “compound” as used herein is meant to include all stereoisomers, geometric isomers, tautomers, and isotopes of the structures named or depicted. Compounds herein identified by name or structure as one particular tautomeric form are intended to include other tautomeric forms unless otherwise specified.

The terms “pharmaceutical” and “pharmaceutically acceptable” are employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans. In some embodiments, the subject is a child (e.g., younger child or older child). In some embodiments, the subject is a child that is 1, 2, 3, 4, 5, 6, 7, 8, 9, or 10 years old. In some embodiments, the subject is a child that is 1-18 years old, 1-16 years old, 1-14 years old, or 1-10 years old.

The term “pediatric” or “pediatric patient” as used herein refers to a patient under the age of 21 years at the time of diagnosis or treatment. The term “pediatric” can be further divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E, Textbook of Pediatrics, 15th Ed. Philadelphia: W.B. Saunders Company, 1996; Rudolph A M, et al., Rudolph's Pediatrics, 21st Ed. New York: McGraw-Hill, 2002; and Avery M D, First L R, Pediatric Medicine, 2nd Ed. Baltimore: Williams & Wilkins; 1994. In some embodiments, a pediatric patient is from birth through the first 28 days of life, from 29 days of age to less than two years of age, from two years of age to less than 12 years of age, or 12 years of age through 21 years of age (up to, but not including, the twenty-second birthday). In some embodiments, a pediatric patient is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

As used herein, the term “pharmaceutically acceptable salt” refers to a salt that is formed between an acid and a basic group of the compound, such as an amino functional group, or a base and an acidic group of the compound, such as a carboxyl functional group. In some embodiments, the compound is a pharmaceutically acceptable acid addition salt. In some embodiments, acids commonly employed to form pharmaceutically acceptable salts of the therapeutic compounds described herein include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfate, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the therapeutic compounds described herein include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C1-C6)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

### Examples

Genomic and molecular profiling of pediatric high-grade gliomas (HGGs) has shown that they are distinct from their adult counterparts.¹⁷⁻¹⁹ The majority of pediatric HGGs, including midline gliomas (e.g., diffuse midline gliomas) with the H3K27M mutation, contain recurrent mutations in histone H3 genes, including H3F3A and HIST1H3B^{6, 20}. These point mutations lead to amino acid substitutions at critical positions in the histone tail resulting in either H3K27M or H3G34R/G34V mutations. Integrated epigenetic and genetic analyses have shown these mutations occur in specific neuroanatomical locations and in different patient populations. The H3K27M mutations are found in the majority of high grade diffuse midline gliomas (thalamic, brainstem, and upper spine), occur in young children, and have rapidly lethal progression; whereas H3G34R/V mutations usually occur in the hemisphere in older children and have a slightly better prognosis.^{6, 21, 22} The H3K27M mutation leads to global hypomethylation of wild-type H3K27 histones and is a critical driver of tumorigenesis in the appropriate cell context and developmental window when combined with other oncogenic mutations, such as p53 loss.²³⁻²⁷ This oncogenic mutation has a dominant negative effect by reprogramming H3K27 methylation and gene expression likely through epigenetic mechanisms including inhibiting the methylating activity of the PRC2 complex.^{15, 23, 24, 26, 27}.

In an effort to find genes that are critically important for H3K27M tumor cell survival and proliferation a focused shRNA knockdown screen was performed to identify gene knockdown targets associated with selective anti-proliferative actions only in H3K27M tumor cells, but not in wild type histone H3 cancer cells. The shRNA library contained ˜1250 shRNAs, targeting most chromatin regulators and major signaling pathways.²⁸ Wnt5a was identified as a candidate gene. To validate whether Wnt5a depletion inhibited proliferation of DIPG cells, Wnt5a was depleted in 4 different tumor lines, two with H3K27M mutations. Depletion of Wnt5a inhibited cell proliferation of the two DIPG lines (SF7761 and SF8628), but had no apparent effect on the proliferation of other lines with wild type H3.3 (SF9427) or H3.3G34V mutation (KNS42) (FIG. 1). Together, these results indicate that Wnt5a is required specifically for proliferation of H3K27M mutant cells. Wnt5a is a secreted glycoprotein that functions in the non-canonical Wnt pathway²⁹. Canonical Wnt signaling involves the binding of a Wnt protein to a family of Frizzled (FZD) receptor proteins and subsequent activation of β-catenin. Wnt5a usually does not promote β-catenin mediated gene transcription and this was verified in the present cell lines by showing that depletion of β-catenin had no effect on DIPG proliferation (FIG. 1, top panel). These results support the hypothesis that Wnt5a inhibits proliferation of DIPG cells through non-canonical Wnt signaling

To date, the role of Wnt5a in DIPG has not been studied. To extend the present findings in patient samples, gene expression of Wnt5a in brain tumors was analyzed with or without H3K27M mutation using published datasets⁷. The expression of Wnt5a was greater than 2-fold higher in DIPG tumors with H3K27M mutation compared to brain tumors with wild type H3 (data not shown). To validate these findings in vivo, Wnt5a was depleted in an orthotopic xenograft model of DIPG tumors and found reduced tumor growth in the Wnt5a knockdowns compared to scrambled shRNA vector or non-treated tumor cells (FIG. 2). These results suggest that high levels of Wnt5a are required for the survival and proliferation of DIPG tumor cells.

Since WNT5a is unlikely a pharmacologically tractable target, a transcription factor reporter screen was used to identify WNT5a signaling networks specifically active in H3K27M mutant DIPG lines. From this screen of 45 transcription factors (QiAGEN) we found that the signal transducer and activator of transcription 3 (STAT3) reporter exhibited high activity in H3K27M cells. Importantly, depletion of Wnt5a expression, while having little effect on the activity of the majority of TFs, significantly reduced the activity of the STAT3 reporter (FIG. 3). These results suggest that STAT3 functions downstream of Wnt5a signaling in DIPG tumor cells. To confirm this, it was evaluated how Wnt5a depletion affects STAT3 phosphorylation. Compared to WT tumor lines, STAT3 phosphorylation was high in the two H3K27M lines (SF7761 and SF8628), and phosphorylation was reduced after Wnt5a depletion in the H3K27M lines, but not in control cell lines (39RG2 and KNS42, FIG. 3). Collectively, these data support the role of STAT3 downstream of Wnt5a signaling in DIPG cells.

The STAT proteins are a family of transcription factors that are activated in response to growth factors and cytokines and promote cell proliferation and survival.³⁰ In normal cells, the activation of STAT proteins is very transient and strictly regulated; however, evidence has shown that some STATs play a key role in oncogenesis.³¹⁻³³ Specifically, activated STAT3 promotes tumorigenesis in a variety of tumors including gliomas.³⁴⁻⁴⁰ Multiple STAT3 pathway inhibitors are in clinical development, and in particular a phase I trial with the brain penetrant STAT inhibitor WP1066 will start soon for adult GBMs.^{35, 36, 41}. (see, e.g., NCT01904123).

The role of STAT3 in midline H3K27M tumors (e.g., diffuse midline H3K27M tumors) has not been studied. To further validate STAT3 signaling as a potential drug target in these patients, it was determined if STAT3 is required for proliferation of H3K27M cells. Depletion of STAT3 using two different shRNA constructs resulted in reduced cell viability of two DIPG cells (SF7761 and SF8628), while having little effect on proliferation of other pediatric glioma lines with wild type H3 (FIG. 4A)—a similar profile to Wnt5a depletion (FIG. 1). Moreover, treatment with WP1066, the STAT3 inhibitor nearing clinical trial,⁴¹ and DJDLeu, another synthetic STAT3 inhibitor,⁴² inhibited cell viability of two H3K27M DIPG cell lines but much less in WT SF9427 cell line (FIG. 4B—WP1066 and 4C—DJDLeu). Because of the critical dependence of STAT3 signaling in H3K27M tumor cells, the activity of STAT3 (pSTAT3) would be elevated in patients with DIPG tumors. Using treatment naive surgical samples from DIPG patients, an increase (>10-fold) in pSTAT3 levels was observed as compared to non-neoplastic brain tissues from other surgeries (FIG. 5A). Using large cohorts from published data sets, it was also seen a >2-fold increase in STAT3 mRNA expression (RNA-seq) in DIPG patients compared to normal brainy (FIG. 5B). In addition, pSTAT3 expression levels were increased in H3K27M cell lines compared to WT lines in both pediatric and adult high-grade gliomas (FIG. 6). Interestingly, pharmacological inhibition of STAT3 function in H3K27M tumor cells increased H3K27me3 levels similar to the H3K27 demethylase inhibitor GSKJ4 (FIGS. 6B and 6C compared to FIG. 6D) supporting the hypothesis that restoring methylation patterns is important for treating H3K27M tumors. The data presented supports a method of treating glioma with H3K27M mutation by restoration of methylation pattern. In one example, the methylation pattern is restored by a H3K27me3 demethylase inhibitor (e.g., GSK-J4).

To validate STAT3 as a therapeutic target for H3K27M tumors, the effects on tumor growth with WP1066 were evaluated in intracranial orthotopic xenografts (FIG. 7). It was found that 8 weeks of oral dosing significantly decreased tumor growth of H3K27M tumor cells compared to control and no toxicities were noted in the mice.

### CONCLUSION

The summation of the results presented in the Examples supports the hypothesis that STAT3 is critical for DIPG cells and is a druggable target. Wnt5a and STAT3 signaling are crucial for proliferation and survival in H3K27M tumor cells.

### REFERENCES

1. Louis D N, Ohgaki H, Wiestler O D, Cavenee W K, Burger P C, Jouvet A, Scheithauer B W, Kleihues P. The 2007 WHO classification of tumours of the central nervous system. Acta neuropathologica. 2007; 114(2):97-109. PMCID: 1929165.
2. Wong E T, Hess K R, Gleason M J, Jaeckle K A, Kyritsis A P, Prados M D, Levin V A, Yung W K. Outcomes and prognostic factors in recurrent glioma patients enrolled onto phase II clinical trials. Journal of clinical oncology: official journal of the American Society of Clinical Oncology. 1999; 17(8):2572-8.
3. Buckner J C. Factors influencing survival in high-grade gliomas. Semin Oncol. 2003; 30(6 Suppl 19):10-4.
4. Jones C, Baker S J. Unique genetic and epigenetic mechanisms driving paediatric diffuse high-grade glioma. Nature reviews Cancer. 2014; 14(10).
5. Wu G, Broniscer A, McEachron T A, Lu C, Paugh B S, Becksfort J, Qu C, Ding L, Huether R, Parker M, Zhang J, Gajjar A, Dyer M A, Mullighan C G, Gilbertson R J, Mardis E R, Wilson R K, Downing J R, Ellison D W, Baker S J. Somatic histone H3 alterations in pediatric diffuse intrinsic pontine gliomas and non-brainstem glioblastomas. Nature genetics. 2012; 44(3):251-3. PMCID: 3288377.
6. Schwartzentruber J, Korshunov A, Liu X Y, Jones D T, Pfaff E, Jacob K, Sturm D, Fontebasso A M, Quang D A, Tonjes M, Hovestadt V, Albrecht S, Kool M, Nantel A, Konermann C, Lindroth A, Jager N, Rausch T, Ryzhova M, Korbel J O, Hielscher T, Hauser P, Garami M, Klekner A, Bognar L, Ebinger M, Schuhmann M U, Scheurlen W, Pekrun A, Fruhwald M C, Roggendorf W, Kramm C, Durken M, Atkinson J, Lepage P, Montpetit A, Zakrzewska M, Zakrzewski K, Liberski P P, Dong Z, Siegel P, Kulozik A E, Zapatka M, Guha A, Malkin D, Felsberg J, Reifenberger G, von Deimling A, Ichimura K, Collins V P, Witt H, Milde T, Witt O, Zhang C, Castelo-Branco P, Lichter P, Faury D, Tabori U, Plass C, Majewski J, Pfister S M, Jabado N. Driver mutations in histone H3.3 and chromatin remodelling genes in paediatric glioblastoma. Nature. 2012; 482(7384):226-31.
7. Sturm D, Witt H, Hovestadt V, Khuong-Quang D A, Jones D T, Konermann C, Pfaff E, Tonjes M, Sill M, Bender S, Kool M, Zapatka M, Becker N, Zucknick M, Hielscher T, Liu X Y, Fontebasso A M, Ryzhova M, Albrecht S, Jacob K, Wolter M, Ebinger M, Schuhmann M U, van Meter T, Fruhwald M C, Hauch H, Pekrun A, Radlwimmer B, Niehues T, von Komorowski G, Durken M, Kulozik A E, Madden J, Donson A, Foreman N K, Drissi R, Fouladi M, Scheurlen W, von Deimling A, Monoranu C, Roggendorf W, Herold-Mende C, Unterberg A, Kramm C M, Felsberg J, Hartmann C, Wiestler B, Wick W, Milde T, Witt O, Lindroth A M, Schwartzentruber J, Faury D, Fleming A, Zakrzewska M, Liberski P P, Zakrzewski K, Hauser P, Garami M, Klekner A, Bognar L, Morrissy S, Cavalli F, Taylor M D, van Sluis P, Koster J, Versteeg R, Volckmann R, Mikkelsen T, Aldape K, Reifenberger G, Collins V P, Majewski J, Korshunov A, Lichter P, Plass C, Jabado N, Pfister S M. Hotspot mutations in H3F3A and IDH1 define distinct epigenetic and biological subgroups of glioblastoma. Cancer cell. 2012; 22(4):425-37.
8. Chan K M, Fang D, Gan H, Hashizume R, Yu C, Schroeder M, Gupta N, Mueller S, James C D, Jenkins R, Sarkaria J, Zhang Z. The histone H3.3K27M mutation in pediatric glioma reprograms H3K27 methylation and gene expression. Genes & development. 2013; 27(9):985-90. PMCID: 3656328.
9. Lewis P W, Muller M M, Koletsky M S, Cordero F, Lin S, Banaszynski L A, Garcia B A, Muir T W, Becher O J, Allis C D. Inhibition of PRC2 activity by a gain-of-function H3 mutation found in pediatric glioblastoma. Science. 2013; 340(6134):857-61.
10. Bender S, Tang Y, Lindroth A M, Hovestadt V, Jones D T, Kool M, Zapatka M, Northcott P A, Sturm D, Wang W, Radlwimmer B, Hojfeldt J W, Truffaux N, Castel D, Schubert S, Ryzhova M, Seker-Cin H, Gronych J, Johann P D, Stark S, Meyer J, Milde T, Schuhmann M, Ebinger M, Monoranu C M, Ponnuswami A, Chen S, Jones C, Witt O, Collins V P, von Deimling A, Jabado N, Puget S, Grill J, Helin K, Korshunov A, Lichter P, Monje M, Plass C, Cho Y J, Pfister S M. Reduced H3K27me3 and DNA hypomethylation are major drivers of gene expression in K27M mutant pediatric high-grade gliomas. Cancer cell. 2013; 24(5):660-72.
11. Venneti S, Garimella M T, Sullivan L M, Martinez D, Huse J T, Heguy A, Santi M, Thompson C B, Judkins A R. Evaluation of histone 3 lysine 27 trimethylation (H3K27me3) and enhancer of Zest 2 (EZH2) in pediatric glial and glioneuronal tumors shows decreased H3K27me3 in H3F3A K27M mutant glioblastomas. Brain pathology. 2013; 23(5):558-64. PMCID: 3701028.
12. Bender S, Tang Y, Lindroth A M, Hovestadt V, Jones D T W, Kool M, Zapatka M, Northcott P A, Sturm D, Wang W, Radlwimmer B, Hojfeldt J W, Truffaux N, Castel D, Schubert S, Ryzhova M, Seker-Cin H, Gronych J, Johann P D, Stark S, Meyer J, Milde T, Schuhmann M, Ebinger M, Monoranu C-M, Ponnuswami A, Chen S, Jones C, Witt O, Collins V P, von Deimling A, Jabado N, Puget S, Grill J, Helin K, Korshunov A, Lichter P, Monje M, Plass C, Cho Y-J, Pfister S M. Reduced H3K27me3 and DNA Hypomethylation Are Major Drivers of Gene Expression in K27M Mutant Pediatric High-Grade Gliomas. Cancer cell. 2013; 24(5):660-72.
13. Castel D, Philippe C, Calmon R, Le Dret L, Truffaux N, Boddaert N, Pages M, Taylor K R, Saulnier P, Lacroix L, Mackay A, Jones C, Sainte-Rose C, Blauwblomme T, Andreiuolo F, Puget S, Grill J, Varlet P, Debily M-A. Histone H3F3A and HIST1H3B K27M mutations define two subgroups of diffuse intrinsic pontine gliomas with different prognosis and phenotypes. Acta Neuropathologica. 2015:1-13.
14. Castel D, Philippe C, Calmon R, Le Dret L, Truffaux N, Boddaert N, Pages M, Taylor K R, Saulnier P, Lacroix L, Mackay A, Jones C, Sainte-Rose C, Blauwblomme T, Andreiuolo F, Puget S, Grill J, Varlet P, Debily M A. Histone H3F3A and HIST1H3B K27M mutations define two subgroups of diffuse intrinsic pontine gliomas with different prognosis and phenotypes. Acta neuropathologica. 2015; 130(6):815-27. PMCID: 4654747.
15. Mohammad F, Weissmann S, Leblanc B, Pandey D P, Hojfeldt J W, Comet I, Zheng C, Johansen J V, Rapin N, Porse B T, Tvardovskiy A, Jensen O N, Olaciregui N G, Lavarino C, Sunol M, de Tones C, Mora J, Carcaboso A M, Helin K. EZH2 is a potential therapeutic target for H3K27M-mutant pediatric gliomas. Nature medicine. 2017.
16. Behjati S, Tarpey P S, Presneau N, Scheipl S, Pillay N, Van Loo P, Wedge D C, Cooke S L, Gundem G, Davies H, Nik-Zainal S, Martin S, McLaren S, Goody V, Robinson B, Butler A, Teague J W, Halai D, Khatri B, Myklebost O, Baumhoer D, Jundt G, Hamoudi R, Tirabosco R, Amary M F, Futreal P A, Stratton M R, Campbell P J, Flanagan A M. Distinct H3F3A and H3F3B driver mutations define chondroblastoma and giant cell tumor of bone. Nature genetics. 2013; 45(12):1479-82. PMCID: 3839851.
17. Faury D, Nantel A, Dunn S E, Guiot M-C, Haque T, Hauser P, Garami M, Bognár L, Hanzely Z, Liberski P P, Lopez-Aguilar E, Valera E T, Tone L G, Carret A-S, Del Maestro R F, Gleave M, Montes J-L, Pietsch T, Albrecht S, Jabado N. Molecular profiling identifies prognostic subgroups of pediatric glioblastoma and shows increased YB-1 expression in tumors. Journal of clinical oncology: official journal of the American Society of Clinical Oncology. 2007; 25(10):1196-208.
18. Paugh B S, Qu C, Jones C, Liu Z, Adamowicz-Brice M, Zhang J, Bax D A, Coyle B, Barrow J, Hargrave D, Lowe J, Gajjar A, Zhao W, Broniscer A, Ellison D W, Grundy R G, Baker S J. Integrated molecular genetic profiling of pediatric high-grade gliomas reveals key differences with the adult disease. Journal of clinical oncology: official journal of the American Society of Clinical Oncology. 2010; 28(18):3061-8. PMCID: PMC2903336.
19. Bax D A, Mackay A, Little S E, Carvalho D, Viana-Pereira M, Tamber N, Grigoriadis A E, Ashworth A, Reis R M, Ellison D W, Al-Sarraj S, Hargrave D, Jones C. A distinct spectrum of copy number aberrations in pediatric high-grade gliomas. Clin Cancer Res. 2010; 16(13):3368-77. PMCID: PMC2896553.
20. Wu G, Broniscer A, McEachron T A, Lu C, Paugh B S, Becksfort J, Qu C, Ding L, Huether R, Parker M, Zhang J, Gajjar A, Dyer M A, Mullighan C G, Gilbertson R J, Mardis E R, Wilson R K, Downing J R, Ellison D W, Baker S J. Somatic histone H3 alterations in pediatric diffuse intrinsic pontine gliomas and non-brainstem glioblastomas. Nat Genet. 2012; 44(3):251-3. PMCID: 3288377.
21. Sturm D, Witt H, Hovestadt V, Khuong-Quang D-A, Jones D T W, Konermann C, Pfaff E, Tönjes M, Sill M, Bender S, Kool M, Zapatka M, Becker N, Zucknick M, Hielscher T, Liu X-Y, Fontebasso A M, Ryzhova M, Albrecht S, Jacob K, Wolter M, Ebinger M, Schuhmann M U, Van Meter T, Fruhwald M C, Hauch H, Pekrun A, Radlwimmer B, Niehues T, von Komorowski G, Durken M, Kulozik A E, Madden J, Donson A, Foreman N K, Drissi R, Fouladi M, Scheurlen W, von Deimling A, Monoranu C, Roggendorf W, Herold-Mende C, Unterberg A, Kramm C M, Felsberg J, Hartmann C, Wiestler B, Wick W, Milde T, Witt O, Lindroth A M, Schwartzentruber J, Faury D, Fleming A, Zakrzewska M, Liberski P P, Zakrzewski K, Hauser P, Garami M, Klekner A, Bognar L, Morrissy S, Cavalli F, Taylor M D, van Sluis P, Koster J, Versteeg R, Volckmann R, Mikkelsen T, Aldape K, Reifenberger G, Collins V P, Majewski J, Korshunov A, Lichter P, Plass C, Jabado N, Pfister S M. Hotspot mutations in H3F3A and IDH1 define distinct epigenetic and biological subgroups of glioblastoma. Cancer cell. 2012; 22(4):425-37.
22. Khuong-Quang D-A, Buczkowicz P, Rakopoulos P, Liu X-Y, Fontebasso A M, Bouffet E, Bartels U, Albrecht S, Schwartzentruber J, Letourneau L, Bourgey M, Bourque G, Montpetit A, Bourret G, Lepage P, Fleming A, Lichter P, Kool M, von Deimling A, Sturm D, Korshunov A, Faury D, Jones D T, Majewski J, Pfister S M, Jabado N, Hawkins C. K27M mutation in histone H3.3 defines clinically and biologically distinct subgroups of pediatric diffuse intrinsic pontine gliomas. Acta neuropathologica. 2012; 124(3):439-47. PMCID: PMC3422615.
23. Chan K M, Fang D, Gan H, Hashizume R, Yu C, Schroeder M, Gupta N, Mueller S, James C D, Jenkins R, Sarkaria J, Zhang Z. The histone H3.3K27M mutation in pediatric glioma reprograms H3K27 methylation and gene expression. Genes &amp; Development. 2013; 27(9): 985-90.
24. Chan K M, Han J, Fang D, Gan H, Zhang Z. A lesson learned from the H3.3K27M mutation found in pediatric glioma. Cell Cycle. 2014; 12(16):2546-52.
25. Funato K, Major T, Lewis P W, Allis C D, Tabar V. Use of human embryonic stem cells to model pediatric gliomas with H3.3K27M histone mutation. Science. 2014; 346(6216): 1529-33.
26. Lewis P W, Muller M M, Koletsky M S, Cordero F, Lin S, Banaszynski L A, Garcia B A, Muir T W, Becher O J, Allis C D. Inhibition of PRC2 activity by a gain-of-function H3 mutation found in pediatric glioblastoma. Science. 2013; 340(6134):857-61. PMCID: PMC3951439.
27. Pathak P, Jha P, Purkait S, Sharma V, Suri V, Sharma M C, Faruq M, Suri A, Sarkar C. Altered global histone-trimethylation code and H3F3A-ATRX mutation in pediatric GBM. Journal of neuro-oncology. 2015; 121(3):489-97.
28. Wang Z, Zhang H, Liu J, Cheruiyot A, Lee J H, Ordog T, Lou Z, You Z, Zhang Z. USP51 deubiquitylates H2AK13,15ub and regulates DNA damage response. Genes & development. 2016; 30(8):946-59. PMCID: 4840300.
29. Nishita M, Enomoto M, Yamagata K, Minami Y. Cell/tissue-tropic functions of Wnt5a signaling in normal and cancer cells. Trends Cell Biol. 2010; 20(6):346-54.
30. Yu H, Kortylewski M, Pardoll D. Crosstalk between cancer and immune cells: role of STAT3 in the tumour microenvironment. Nat Rev Immunol. 2007; 7(1):41-51.
31. Yu C, Meyer D, Campbell G, Lamer A, Carter-Su C, Schwartz J, Jove R. Enhanced DNA-binding activity of a Stat3-related protein in cells transformed by the Src oncoprotein. Science. 1995; 269(5220):81-3.
32. Yu H, Jove R. The STATs of cancer—new molecular targets come of age. Nat Rev Cancer. 2004; 4(2):97-105.
33. Bromberg J F, Wrzeszczynska M R, Devgan G, Zhao Y, Pestell R G, Albanese C, Darnell J E. Stat3 as an oncogene. Cell. 1999; 98(3):295-303.
34. Bowman T, Garcia R, Turkson J, Jove R. STATs in oncogenesis. Oncogene. 2000; 19(21):2474-88.
35. Heimberger A. The therapeutic potential of inhibitors of the signal transducer and activator of transcription 3 for central nervous system malignancies. Surgical neurology international. 2011; 2(1):163.
36. Iwamaru A, Iwamaru A, Szymanski S, Szymanski S, Iwado E, Iwado E, Aoki H, Aoki H, Yokoyama T, Yokoyama T, Fokt I, Fokt I, Hess K, Hess K, Conrad C, Conrad C, Madden T, Madden T, Sawaya R, Sawaya R, Kondo S, Kondo S, Priebe W, Priebe W, Kondo Y, Kondo Y. A novel inhibitor of the STAT3 pathway induces apoptosis in malignant glioma cells both in vitro and in vivo. Oncogene. 2007; 26(17):2435-44.
37. Kim J, Patel M, Ruzevick J, Jackson C, Lim M. STAT3 Activation in Glioblastoma: Biochemical and Therapeutic Implications. Cancers. 2014; 6(1):376-95.
38. Schust J, Sperl B, Hollis A, Mayer T U, Berg T. ScienceDirect.com—Chemistry &amp; Biology—Stattic: A Small-Molecule Inhibitor of STAT3 Activation and Dimerization. Chemistry &amp; Biology. 2006; 13(11):1235-42.
39. Stechishin O D, Luchman H A, Ruan Y, Blough M D, Nguyen S A, Kelly J J, Cairncross J G, Weiss S. On-target JAK2/STAT3 inhibition slows disease progression in orthotopic xenografts of human glioblastoma brain tumor stem cells. Neuro-oncology. 2013; 15(2):198-207.
40. Yu H, Pardoll D, Jove R. STATs in cancer inflammation and immunity: a leading role for STAT3. 2009:1-12.
41. Muhammad AKMG, Puntel M, Candolfi M, Salem A, Yagiz K, Farrokhi C, Kroeger K M, Xiong W, Curtin J F, Liu C, Lawrence K, Bondale N S, Lerner J, Baker G J, Foulad D, Pechnick R N, Palmer D, Ng P, Lowenstein P R, Castro M G. Study of the Efficacy, Biodistribution, and Safety Profile of Therapeutic Gutless Adenovirus Vectors as a Prelude to a Phase I Clinical Trial for Glioblastoma. Clin Pharmacol Ther. 2010; 88(2):204-13.
42. http://www.ascopost.com/News/44159.
43. Paugh B S, Broniscer A, Qu C, Miller C P, Zhang J, Tatevossian R G, Olson J M, Geyer J R, Chi S N, da Silva N S, Onar-Thomas A, Baker J N, Gajjar A, Ellison D W, Baker S J. Genome-wide analyses identify recurrent amplifications of receptor tyrosine kinases and cell-cycle regulatory genes in diffuse intrinsic pontine glioma. Journal of clinical oncology: official journal of the American Society of Clinical Oncology. 2011; 29(30):3999-4006. PMCID: 3209696.
44. Harris L W, Lockstone H E, Khaitovich P, Weickert C S, Webster M J, Bahn S. Gene expression in the prefrontal cortex during adolescence: implications for the onset of schizophrenia. BMC medical genomics. 2009; 2:28. PMCID: 2694209.
45. Carey B W, Finley L W S, Cross J R, Allis C D, Thompson C B. Intracellular alpha-ketoglutarate maintains the pluripotency of embryonic stem cells. Nature. 2015; 518(7539):413-6.
46. Hashizume R, Andor N, Ihara Y, Lerner R, Gan H, Chen X, Fang D, Huang X, Tom M W, Ngo V, Solomon D, Mueller S, Paris P L, Zhang Z, Petritsch C, Gupta N, Waldman T A, James C D. Pharmacologic inhibition of histone demethylation as a therapy for pediatric brainstem glioma. Nature medicine. 2014; 20(12):1394-6. PMCID: 4257862.
47. Vermes I, Haanen C, Steffens-Nakken H, Reutelingsperger C. A novel assay for apoptosis. Flow cytometric detection of phosphatidylserine expression on early apoptotic cells using fluorescein labelled Annexin V. J Immunol Methods. 1995; 184(1):39-51.
48. Nadkarni A, Shrivastav M, Mladek A C, Schwingler P M, Grogan P T, Chen J, Sarkaria J N. ATM inhibitor KU-55933 increases the TMZ responsiveness of only inherently TMZ sensitive GBM cells. Journal of neuro-oncology. 2012; 110(3):349-57. PMCID: 3535329.
49. Cecchini M J, Amiri M, Dick F A. Analysis of cell cycle position in mammalian cells. Journal of visualized experiments: JoVE. 2012(59). PMCID: 3462567.
50. Love M I, Huber W, Anders S. Moderated estimation of fold change and dispersion for RNA-seq data with DESeq2. Genome Biol. 2014; 15(12).
51. Li H, Durbin R. Fast and accurate long-read alignment with Burrows-Wheeler transform. Bioinformatics. 2010; 26(5):589-95.
52. Zhang Y, Liu T, Meyer C A, Eeckhoute J, Johnson D S, Bernstein B E, Nussbaum C, Myers R M, Brown M, Li W, Liu X S. Model-based Analysis of ChIP-Seq (MACS). Genome Biol. 2008; 9(9).
53. Robinson M D, Smyth G K. Small-sample estimation of negative binomial dispersion, with applications to SAGE data. Biostatistics. 2008; 9(2):321-32.
54. Qin J, Hu Y H, Xu F, Yalamanchili H K, Wang J W. Inferring gene regulatory networks by integrating ChIP-seq/chip and transcriptome data via LASSO-type regularization methods. Methods. 2014; 67(3):294-303.
55. VanderVeen N, Paran C, Krasinkiewicz J, Zhao L, Palmer D, Hervey-Jumper S, Ng P, Lowenstein P R, Castro M G. Effectiveness and Preclinical Safety Profile of Doxycycline to Be Used “Off-Label” to Induce Therapeutic Transgene Expression in a Phase I Clinical Trial for Glioma. Hum Gene Ther Cl Dev. 2013; 24(3):116-26.
56. Muhammad AKMG, Xiong W D, Puntel M, Farrokhi C, Kroeger K M, Salem A, Lacayo L, Pechnick R N, Kelson K R, Palmer D, Ng P, Liu C Y, Lowenstein P R, Castro M G. Safety Profile of Gutless Adenovirus Vectors Delivered into the Normal Brain Parenchyma: Implications for a Glioma Phase 1 Clinical Trial. Hum Gene Ther Method. 2012; 23(4):271-84.
57. Funato K, Major T, Lewis P W, Allis C D, Tabar V. Use of human embryonic stem cells to model pediatric gliomas with H3.3K27M histone mutation. Science. 2014; 346(6216): 1529-33.
58. Halvorson K G, Barton K L, Schroeder K, Misuraca K L, Hoeman C, Chung A, Crabtree D M, Cordero F J, Singh R, Spasojevic I, Berlow N, Pal R, Becher O J. A high-throughput in vitro drug screen in a genetically engineered mouse model of diffuse intrinsic pontine glioma identifies BMS-754807 as a promising therapeutic agent. PloS one. 2015; 10(3):e0118926. PMCID: 4352073.
59. Grasso C S, Tang Y, Truffaux N, Berlow N E, Liu L, Debily M A, Quist M J, Davis L E, Huang E C, Woo P J, Ponnuswami A, Chen S, Johung T B, Sun W, Kogiso M, Du Y, Qi L, Huang Y, Hutt-Cabezas M, Warren K E, Le Dret L, Meltzer P S, Mao H, Quezado M, van Vuurden D G, Abraham J, Fouladi M, Svalina M N, Wang N, Hawkins C, Nazarian J, Alonso M M, Raabe E H, Hulleman E, Spellman P T, Li X N, Keller C, Pal R, Grill J, Monje M. Functionally defined therapeutic targets in diffuse intrinsic pontine glioma. Nature medicine. 2015.
60. Misuraca K L, Cordero F J, Becher O J. Pre-Clinical Models of Diffuse Intrinsic Pontine Glioma. Frontiers in oncology. 2015; 5.
61. Bordiuk O L, Smith K, Morin P J, Semenov M V. Cell Proliferation and Neurogenesis in Adult Mouse Brain. PloS one. 2014; 9(11).
62. Taylor J W, Dietrich J, Gerstner E R, Norden A D, Rinne M L, Cahill D P, Stemmer-Rachamimov A, Wen P Y, Betensky R A, Giorgio D H, Snodgrass K, Randall A E, Batchelor T T, Chi A S. Phase 2 study of bosutinib, a Src inhibitor, in adults with recurrent glioblastoma. Journal of neuro-oncology. 2015; 121(3):557-63. PMCID: 4323868.
63. Puget S, Blauwblomme T, Grill J. Is biopsy safe in children with newly diagnosed diffuse intrinsic pontine glioma? American Society of Clinical Oncology educational book American Society of Clinical Oncology Meeting. 2012:629-33.
64. PNOC. https://clinicaltrials.gov/ct2/show/NCT02960230?term=PNOC&rank=9.
65. UCSF. https://clinicaltrials.gov/ct2/show/NCT02274987?term=DIPG+clinical+trial&rank=4.
66. Skolnik J M, Barrett J S, Jayaraman B, Patel D, Adamson P C. Shortening the timeline of pediatric phase I trials: The rolling six design. Journal of Clinical Oncology. 2008; 26(2): 190-5.
67. Le Tourneau C, Lee J J, Siu L L. Dose Escalation Methods in Phase I Cancer Clinical Trials. J Natl Cancer I. 2009; 101(10):708-20.
68. Fouladi M, Perentesis J P, Wagner L M, Vinks A A, Reid J M, Ahern C, Thomas G, Mercer C A, Krueger D A, Houghton P J, Doyle L A, Chen H, Weigel B, Blaney S M. A Phase I Study of Cixutumumab (IMC-A12) in Combination with Temsirolimus (CCI-779) in Children with Recurrent Solid Tumors: AChildren's Oncology Group Phase I Consortium Report. Clinical Cancer Research. 2015; 21(7):1558-65.
69. Loh M L, Tasian S K, Rabin K R, Brown P, Magoon D, Reid J M, Chen X J, Ahern C H, Weigel B J, Blaney S M. A phase 1 dosing study of ruxolitinib in children with relapsed or refractory solid tumors, leukemias, or myeloproliferative neoplasms: A Children's Oncology Group phase 1 consortium study (ADVL1011). Pediatric blood & cancer. 2015; 62(10):1717-24.
70. DuBois S G, Marachelian A, Fox E, Kudgus R A, Reid J M, Groshen S, Malvar J, Bagatell R, Wagner L, Maris J M, Hawkins R, Courtier J, Lai H, Goodarzian F, Shimada H, Czarnecki S, Tsao-Wei D, Matthay K K, Mosse Y P. Phase I Study of the Aurora A Kinase Inhibitor Alisertib in Combination With Irinotecan and Temozolomide for Patients With Relapsed or Refractory Neuroblastoma: A NANT (New Approaches to Neuroblastoma Therapy) Trial. Journal of Clinical Oncology. 2016; 34(12): 1368-+.
71. Pokorny J L, Calligaris D, Gupta S K, Iyekegbe D O, Jr., Mueller D, Bakken K K, Carlson B L, Schroeder M A, Evans D L, Lou Z, Decker P A, Eckel-Passow J E, Pucci V, Ma B, Shumway S D, Elmquist W F, Agar N Y, Sarkaria J N. The Efficacy of the Wee1 Inhibitor MK-1775 Combined with Temozolomide Is Limited by Heterogeneous Distribution across the Blood-Brain Barrier in Glioblastoma. Clinical cancer research: an official journal of the American Association for Cancer Research. 2015; 21(8):1916-24. PMCID: 4401631.

### Other Embodiments

It is to be understood that while the present application has been described in conjunction with the detailed description thereof, the foregoing description is intended to illustrate and not limit the scope of the present application, which is defined by the scope of the appended claims. Other aspects, advantages, and modifications are within the scope of the following claims.

1. A method of treating a malignant glioma in a subject, the method comprising:
a) identifying a mutation in a histone H3 gene in a glioma cell obtained from the subject; and
b) after a), administering to the subject a therapeutically effective amount of a STAT3 inhibitor, or a pharmaceutically acceptable salt thereof.

2. The method of claim 1, wherein the glioma is pediatric.

3. The method of claim 1, wherein the glioma is selected from a high-grade glioma (HGG), midline glioma, diffuse midline glioma, thalamic glioma, brainstem glioma, upper spine glioma, and Diffuse Intrinsic Pontine Glioma (DIPG).

4-7. (canceled)

8. The method of claim 1, wherein the histone H3 gene is H3F3A.

9. The method of claim 8, wherein the mutation leads to lysine (K) substitution with methionine (M) or isoleucine (I) in the histone tail.

10-12. (canceled)

13. The method of claim 8, wherein the mutation in the histone H3 gene results in a translation of a H3 histone having a K27M amino acid substitution (H3K27M mutation).

14. The method of claim 8, wherein the mutation in the histone H3 gene results in a translation of a H3 histone having a K27I amino acid substitution (H3K27I mutation).

15. The method of claim 1, wherein the mutation leads to global hypomethylation of H3 histones in the glioma.

16. The method of claim 15, wherein the mutation leads to decreased levels or global loss of H3K27me3 and/or H3K27me2 in the glioma.

17. A method of treating Diffuse Intrinsic Pontine Glioma (DIPG) in a subject, the method comprising administering to the subject a therapeutically effective amount of a STAT3 inhibitor, or a pharmaceutically acceptable salt thereof.

18. The method of claim 17, wherein the Diffuse Intrinsic Pontine Glioma (DIPG) is pediatric.

19. The method of claim 1, wherein the STAT3 inhibitor, or a pharmaceutically acceptable salt thereof, is administered to the subject orally.

20. The method of claim 1, wherein the STAT3 inhibitor, or a pharmaceutically acceptable salt thereof, is a blood brain barrier penetrant.

21. The method of claim 1, wherein the STAT3 inhibitor is WP1066 having the following structure: [chemical structure]
or a pharmaceutically acceptable salt thereof.

22. The method of claim 1, wherein the STAT3 inhibitor is S3I-201 having the following structure: [chemical structure]
or a pharmaceutically acceptable salt thereof.

23. The method of claim 1, wherein the STAT3 inhibitor is C10 having the following structure: [chemical structure]
or a pharmaceutically acceptable salt thereof.

24. The method of claim 1, wherein the STAT3 inhibitor is selected from any one of the following compounds (C1-C9): [chemical structure]
or a pharmaceutically acceptable salt thereof.

25. The method of claim 1, wherein the STAT3 inhibitor directs dephosphorylation and nuclear export of constitutively phosphorylated STAT3.

26. The method of claim 1, wherein the administration of STAT3 inhibitor to the subject leads to an increased level of a methylated H3 histone in the glioma, and the increased level of the methylated H3 histone results in the treatment of the glioma in the subject.

27. The method of claim 26, wherein the methylated H3 histone is H3K27me2 and/or H3K27me2.

28. (canceled)
